(19) United States
(12) Patent Application Publication
(10) Pub. No.: US 2008/0090261 A1
(43) Pub. Date: Apr. 17, 2008

(54) Method For The In Vitro Diagnosis Of The Exclusion Of Acute Coronary Syndromes

(75) Inventors: Eric Bonnefoy, Chassieu (FR); Gordon Lowe, Glasgow (GB)

Correspondence Address: OLIFF & BERRIDGE, PLC, P.O. BOX 320850, ALEXANDRIA, VA, 22320-4850, US

(73) Assignee: BIOMERIEUX, Marcy L'etoile (FR)

(21) Appl. No.: 11/662,055

(22) PCT Filed: Oct. 10, 2005
(86) PCT No.: PCT/FR05/50835

§ 371 Date: Mar. 7, 2007

(30) Foreign Application Priority Data
Oct. 11, 2004 (FR) 0452335

(51) Int. Cl. C12Q 1/02; G01N 33/00; G01N 33/53

(52) U.S. Cl. 435/7.92; 435/29; 435/7.1

(57) ABSTRACT

The present invention relates to a method for the in vitro diagnosis of the exclusion of acute coronary syndromes (ACS), consisting in associating the quantification of the concentration of at least one cardiac marker selected from troponin, CK-MB and myoglobin and the quantification of the concentration of D-dimers in a sample, and to the use of a reagent for quantifying the concentration of at least one of said cardiac markers and of a reagent for quantifying the concentration of D-dimers for the in vitro diagnosis of the exclusion of an acute coronary syndrome.

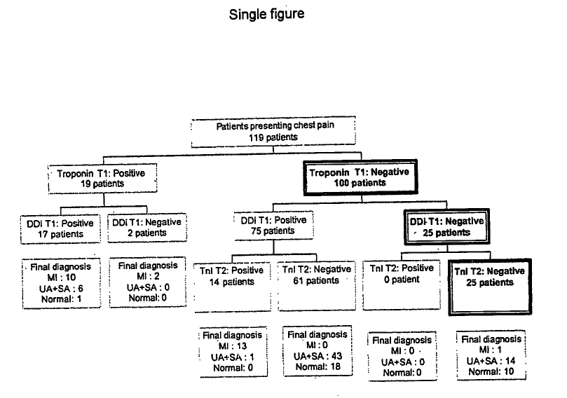

The present invention relates to a method for the in vitro diagnosis of the exclusion of acute coronary syndromes (ACS), commonly called myocardial infarction (MI), in patients presenting chest pain and suspected of being at risk, according to which the quantification of the concentration of at least one cardiac marker selected from troponin, CK-MB and myoglobin and the quantification of the concentration of D-dimers, based on a sample, are associated.

Myocardial infarction is most commonly due to an abrupt occlusive thrombosis of a coronary artery, resulting in ischemic necrosis of a more or less extensive area of the cardiac muscle. This acute coronary occlusion due to a thrombus is most commonly associated with fissuration or rupture of an atheroma plaque.

Myocardial infarction is a disease with a considerable risk of fatality in the short or medium term and constitutes an absolute cardiological emergency, the incidence of which is still very high throughout the world. Myocardial infarction most commonly manifests itself at night or while resting, through intense pain that appears abruptly in the chest, behind the sternum. This pain resembles that of angina pectoris. In practice, patients suffering from acute chest pain must be transported as rapidly as possible to emergency departments or EDs, in which, when the patient is admitted, a series of examinations and tests is carried out, including a study of the patient's clinical history, an electrocardiogram and the assaying of cardiac markers, such as troponin and/or CK-MB and/or myoglobin. However, the assaying of cardiac enzymes, CK-MB and myoglobin, has the drawback of not being specific for the pathology, and practitioners have to carry out other specialized examinations, such as continuous electrocardiogram, or imaging techniques, such as cardiac Doppler echography and coronography, before putting forward a diagnosis. Such specialized examinations are not available in all hospitals, and rarely 24 hours a day. As regards the assaying of troponin, since several hours may elapse during a myocardial infarction before the troponin concentration becomes elevated, and since the latter is only detectable in the blood six hours after the beginning of the chest pain, a further assaying of tropinin is required four to six hours after the first troponin assay in order to exclude a myocardial infarction in patients who present a normal electrocardiogram, in accordance with the NACB guidelines [“NACB Laboratory Medicine Practice Guidelines: Characteristics and Utilization of Biochemical Markers in ACS and Heart Failure, CLINICAL: ACUTE CORONARY SYNDROMES—Chapter 1, 2004”].

There exists therefore today a real need to establish a very early strategy for excluding patients suspected of acute coronary syndrome when they are admitted to an emergency department. A new strategy would make it possible to limit the additional enzyme assays and/or the supplementary examinations which represent an additional cost, to avoid wasting time, to improve the patient's comfort and possibly to direct said patient to the appropriate department depending on his or her pathology. In fact, the earlier the diagnosis, the better the patient's care will be. In such an approach, the negative prediction value and sensitivity are the most important parameters.

In patent application U.S. Pat. No. 6,309,888, it is proposed to distinguish between the acute and nonacute stages of coronary syndromes by searching for (1) a first marker, such as malondialdehyde-modified LDLs, the presence of which above a predetermined level indicates a very high degree of diagnostic precision (i.e. the surface area under the ROC or “Receiver Operating Characteristics” curve is at least 0.875) for the presence of an acute stage of ACS, then (2) either a second marker, such as oxidized LDLs, the presence of which above a predetermined level indicates a very high degree of diagnostic precision for the presence of ACS, (2′) or a third marker, such as troponin I, the presence of which above a predetermined level indicates a high degree of diagnostic precision (i.e. the surface area under the ROC or “Receiver Operating Characteristics” curve is at least 0.70) for the presence of an acute stage of ACS, (2″) or the second and the third markers as defined above.

Surprisingly, the applicant has found that associating the quantification of the concentration of at least one cardiac marker selected from troponin, CK-MB and myoglobin and the quantification of the concentration of D-dimers, based on a liquid sample from a patient presenting chest pain, makes it possible to exclude the diagnosis of an acute coronary syndrome with a high sensitivity and a high negative prediction value, which can reach up to approximately 100%, which enables both a reduction in investigation costs and in time and an acceleration of the approaches for shedding light on the differential diagnosis.

A subject of the present invention is therefore a method for the in vitro diagnosis of the exclusion of an acute coronary syndrome based on a liquid sample from a patient suspected of presenting a risk, according to which:
the concentration of at least one cardiac marker selected from troponin, CK-MB and myoglobin, in said sample, is quantified,
the concentration of D-dimers in said sample is quantified,
the concentration of said cardiac marker(s) of the sample is compared to a predetermined threshold concentration,
the concentration of D-dimers of the sample is compared to a predetermined threshold concentration of D-dimers, and
it is determined whether the concentration of said cardiac marker(s) and the concentration of D-dimers of the sample are respectively less than the predetermined threshold concentration of said cardiac marker(s) and than the predetermined threshold concentration of D-dimers;
   it being understood that the quantification of the concentration of said cardiac marker(s) and the quantification of the concentration of D-dimers are carried out either sequentially and independently of the order, or simultaneously, as explained in greater detail below, based on the example of the quantification of the concentration of troponin and the quantification of the concentration of D-dimers.

According to the method of the invention, it is therefore excluded that a patient is suffering from an acute coronary syndrome when the concentrations of cardiac marker(s) and of D-dimers are less than the predetermined threshold concentrations.

In one embodiment of the invention,
the concentration of troponin in said sample is quantified,
the concentration of D-dimers in said sample is quantified,
the concentration of troponin of the sample is compared to a predetermined threshold concentration of troponin,
the concentration of D-dimers of the sample is compared to a predetermined threshold concentration of D-dimers, and
it is determined whether the concentration of troponin and the concentration of D-dimers of the sample are respectively less than the predetermined threshold concentration of troponin and than the predetermined threshold concentration of D-dimers.

According to the method of the invention, it is possible to exclude the diagnosis of an ACS, based on a liquid sample from a patient presenting chest pain, with a sensitivity and a negative prediction value that are superior to the performance levels of the isolated initial assaying of cardiac markers such as troponin when the patient presents at the emergency department.

In fact, to date, although the performance level of the new generations of troponin assays has improved, the sensitivities and the negative prediction values do not reach values in the region of 100%.

The quantification of the concentration of said cardiac marker(s) and of the D-dimers can be carried out by any method known to those skilled in the art for determining a concentration of a marker in a liquid sample. This quantification is preferably carried out using at least one antibody specific for said cardiac marker(s) or for the D-dimers, or at least one antibody fragment specific for said cardiac marker(s). Preferably, at least one monoclonal antibody is used, for instance an anti-troponin monoclonal antibody. The quantification of D-dimers is preferably carried out using at least one anti-D-dimer antibody or at least one anti-D-dimer antibody fragment. Preferably, at least one anti-D-dimer monoclonal antibody is used.

The term “antibody fragment” is intended to mean in particular F(ab)2, Fab, Fab′ or sFv fragments [Blazar et al., 1997, Journal of Immunology 159: 5821-5833 and Bird et al., 1988, Science 242: 423-426] of a natural antibody.

In the rest of the description which follows and in the claims, the generic term “antibody” will be used to denote, without distinction, a monoclonal antibody, a monospecific polyclonal antibody or an antibody fragment.

In one embodiment of the method of the invention, the concentration of cardiac marker, preferably troponin, in the sample is quantified and then the concentration of D-dimers in the sample is quantified.

In a second embodiment of the method of the invention, the concentration of D-dimers in the sample is quantified and then the concentration of cardiac marker, such as troponin, in the sample is quantified.

In a third embodiment of the method of the invention, the concentration of cardiac marker, such as troponin, and the concentration of D-dimers in the sample are simultaneously quantified. In this embodiment, (i) at least one anti-cardiac marker antibody (for example anti-troponin) and at least one anti-D-dimer antibody, corresponding to the definitions given above, can be attached to one and the same solid phase, it being possible for said solid phase to consist of a tube, a strip or a cone (see, for example, the principle of the VIDAS® HIV DUO test (reference 30 114) sold by the applicant), or (ii) at least one anti.-cardiac marker antibody (for example, anti-troponin) is attached to one solid phase and at least one anti-D-dimer antibody is attached to another solid phase, said antibodies corresponding to the definitions given above, and it being possible for the solid phase to consist of a tube, a strip, a cone or a particle.

A subject of the invention is also the use of a reagent for quantifying the concentration of at least one cardiac marker selected from troponin, CK-MB and myoglobin, in a liquid sample, and of a reagent for quantifying the concentration of D-dimers in a liquid sample, said reagents being intended for use for the in vitro diagnosis of the exclusion of an acute coronary syndrome.

In one embodiment of the invention, said reagent for quantifying the concentration of said cardiac marker(s) is at least one antibody corresponding to the definitions given above, and said reagent for quantifying the concentration of D-dimers is at least one antibody corresponding to the definitions given above. Preferably, one of the reagents is an anti-troponin antibody and the other reagent is an anti-D-dimer antibody.

The liquid sample is any liquid sample from the human body that may contain the cardiac markers and the D-dimers. Preferably, it is selected from serum, plasma and blood.

Troponin is one of the protein components of striated muscle. It is made up of three subunits, I, T and C, which are involved in muscle contraction. Troponin I and troponin T make it possible to differentiate between a myocardial necrosis and a skeletal muscle attack. One of the three isoforms of troponin, troponin Ic, exhibits cardiac specificity with no cross reaction. In the method of the invention, the concentration of troponin T or the concentration of troponin I, and preferably the concentration of troponin Ic, is quantified.

Creatinin kinase, CK, is a dimer consisting of two subunits denoted M and B, combined to give three isoenzymes: CK-BB, CK-MB and CK-MM. These three isoenzymes are located in the cytoplasm and each has a molecular weight of approximately 82 000 daltons. The CK in the serum of a normal adult man consists mainly of the CK-MM isoenzyme with only trace amounts of CK-MB. The CK-BB isoenzyme is not usually present in the serum at the limits of detection in most CK assays. The detection of significant amounts of CK-MB in the serum is usually indicative of ACS. However, CK-MB has also been found in the serum of patients exhibiting disorders other than ACS.

Myoglobin is a low-molecular-weight protein derived from the heme of muscle cells. It constitutes a marker for irreversible cell necrosis, the serum level of which increases early on after the beginning of infarction. Its half-life, which is very brief, allows it to closely reflect the evolution of the infarction. In a population of normal individuals, the blood myoglobin level is from 6 to 85 ng/ml. The diagnostic threshold is set at between 70 and 90 ng/ml. In the course of an MI, myoglobin appears between the 2^{nd} and the 4^{th} hour, with a peak between the 9^{th} and the 12^{th} hours. It remains assayable for from 7 to 20 hours after the peak. The proportion of false negatives depends on the amount of time between admission and the beginning of chest pain and is from 45% to 67% before the 4^{th} hour. Myoglobin exists in all the striated muscles of the organism. The false positives are therefore the procedures carried out for cardiac reanimation, external electric shocks, intramuscular injections, and certain myopathies. The increase is usually relatively insubstantial and the context often makes it possible to identify them. The myoglobin level also increases in the event of renal insufficiency and in any situation which induces a decrease in glomerular filtration, such as advanced cardiac insufficiency. Age, weight and sex have little influence on blood myoglobin levels. Similarly, blood myoglobin levels usually remain normal after exercise.

D-dimers are products of fibrin degradation, soluble fragments of very heterogeneous composition that result from two simultaneous phenomena:
coagulation of fibrinogen to stabilized fibrin, after the action of thrombin and of factor XIIIa,
digestion of the fibrin clot, by plasmin, to soluble fragments that are released into the bloodstream. The end products of the lysis of the clot are the D-dimers.

Among all the markers of thrombotic states, D-dimers are the only ones that really attest to the presence of stabilized fibrin.

Said cardiac marker(s), in particular troponin, and the D-dimers can be quantified using various immunoassay principles which are well known to those skilled in the art, such as ELISA, ELFA, latex agglutination, turbidimetry, turbidimetry with particles, nephelometry, nephelometry with particles, or other appropriate methods. These principles and other methods are, for example, described in “Labor und Diagnose”, ed. L. Thomas, TH-Books Verlagsgesellschaft mbH, Frankfurt, 1998, chapter 60 or in “Laboratory Techniques in Biochemistry and Molecular Biology,—An introduction to Radioimmunoassay and Related Techniques” ed. T. Chard, Elsevier, Amsterdam, 1987.

Thus, the quantification of the concentration of said cardiac marker(s), in particular of troponin, and the quantification of the concentration of D-dimers in the sample are carried out, independently of one another, by means of an immunoassay, on the basis of an assay selected from ELISA, ELFA, turbidimetry, nephelometry, turbidimetry with particles, nephelometry with particles, and latex agglutination.

The threshold concentration values can be defined by those skilled in the art, individually for the cardiac markers such as troponin, and the D-dimers, on the basis of well-known procedures. By way of example, the following procedure can be applied for the D-dimers: the samples from a number of patients diagnosed as ACS-positive, for example by imaging, and a number of samples from ACS-negative individuals, are assayed for the concentration of D-dimers; the results obtained are evaluated on the basis of various threshold values and the threshold concentration values that make it possible to exclude the ACS-negatives and that do not make it possible to exclude the ACS-positives are selected. The threshold concentration values may vary according to the technique and the kit used for the D-dimer assay. This procedure is also applicable to the cardiac markers.

In the specific case of troponin, the threshold is determined in accordance with international recommendations and must correspond to the 99^{th} percentile of a reference control population [“NACB Laboratory Medicine Practice Guidelines: Characteristics & Utilization of Biochemical Markers in ACS and Heart Failure, CLINICAL: ACUTE CORONARY SYNDROMES—Chapter 1, 2004”]. The threshold concentration values may also vary according to the technique and to the kit used for the troponin assay.

A preferred but nonlimiting embodiment of the present invention is the method for the in vitro diagnosis of the exclusion of an acute coronary syndrome in a patient suspected of presenting a risk, as described above, in which troponin, preferably troponin Ic, is tested using a sandwich immunoenzymatic method on a device such as the VIDAS® instrument, by applying a threshold concentration value of 0.1 μg/l.

Another preferred but nonlimiting embodiment of the present invention is the method for the in vitro diagnosis of the exclusion of an acute coronary syndrome in a patient suspected of presenting a risk, as described above, in which the D-dimers are tested using a sandwich immunoenzymatic method on a device such as the VIDAS® instrument, by applying a threshold concentration value of 250 ng/ml.

The method for the in vitro diagnosis of the exclusion of an ACS, described above, was validated by means of a prospective study, carried out over six months, on 119 patients, from an emergency department, suffering from chest pain. The method of the invention made it possible to exclude the diagnosis of cardiac muscle necrosis with a very high sensitivity and a very high negative predictive value, superior to the performance levels of assaying troponin alone at the time the patient presented at the emergency department.

The method of the invention is described in greater detail below and on the single figure representing the algorithm used to evaluate the combined use of troponin Ic and of D-dimers and to diagnose the patients excluded from ACS. None of these details can be considered to limit the invention in any way whatsoever.

### EXAMPLE

### Materials and Methods

119 consecutive patients (88 men and 31 women) presenting chest pain were selected in the emergency department. The average age was 59 years old, more or less thirteen years, in the study. An electrocardiogram was carried out for all the patients. Fresh plasma samples collected from these patients were tested with the VIDAS® D-Dimer New kit (bioMérieux) and with the VIDAS® Troponin I (TNI) kit (bioMérieux) at different times, on the VIDAS® instrument (bioMérieux).

In the study, the time T0 corresponds to the appearance of the chest pain, the time T1 corresponds to the time at which the first sample was taken when the patient arrived at the emergency department, and the time T2 corresponds to T1 plus 6 hours. A threshold value of 0.1 μg/l was set for the troponin I, in accordance with the new international recommendations (99^{th} percentile of a normal population) for excluding a myocardial infarction. The optimal threshold value for the D-dimers was determined for excluding myocardial infarction. An ROC (“Receiver Operating Characteristics”) curve, for the D-dimers, for predicting a myocardial infarction was established using the Analyse-It program. The ROC curve for excluding myocardial infarction for the D-dimers gives an optimal threshold value at 250 ng/ml (Youden index: 14.3%, sensitivity: 88.5% and specificity: 25.8%; area under the curve 0.60).

The algorithm used to evaluate the combined use of troponin Ic and of D-dimers is described in the single figure, in which DDi=D-dimers, TnI=troponin I, MI=myocardial infarction, UA=unstable angina, SA=stable angina.

It can be seen on this figure that, if the concentration of troponin Ic is greater than 0.1 μg/l at time T1 and if the concentration of D-dimers is less than 250 ng/ml at time T1, myocardial infarction cannot be excluded. If the concentration of troponin Ic is less than 0.1 μg/l at time T1 and if the concentration of D-dimers is less than 250 ng/ml at time T1, myocardial infarction is excluded, in accordance with the method of the invention.

Sensitivity and specificity:

| | D-dimers | Troponin | Troponin + | |
|---|---|---|---|---|
| | (250 ng/ml) | (0.1 μg/l) | D-dimers | Troponin |
| Time | T1 | T1 | T1 | T2 |
| Sensitivity | 88.5% | 46.2% | 96.2% | 96.2% |
| Specificity | 25.8% | 92.5% | 25.8% | 91.4% |

### Discussion

26 patients (22%) are diagnosed MI-positive. Among these patients, just one has a negative troponin and a negative D-dimers at time T1. However, as for the other 25 patients who were troponin-negative and D-dimer-negative at T1, the troponin of said patient remains negative at T2. With the algorithm defined above, 21% ( 25/119) of the population can be excluded in the emergency departments in terms of an MI as soon as the results of the assays for troponin and the D-dimers carried out at time T1 are obtained.

A D-dimers-negative result (<250 ng/ml) associated with a troponin-negative result improves the sensitivity of the diagnosis at time T1, compared to the result obtained with troponin alone, from 46.2% to 96.2%. With the combination of the two tests, the sensitivity obtained at time T1 is equal to that obtained with the troponin assay alone at time T2.

The association of the troponin and D-dimer assays is a combination that allows a very early exclusion of patients suspected of presenting a risk of MI in emergency departments, with a very high sensitivity and a very high negative prediction value.

1. A method for the in vitro diagnosis of the exclusion of an acute coronary syndrome based on a liquid sample from a patient suspected of presenting a risk, according to which:
the concentration of at least one cardiac marker selected from troponin, CK-MB and myoglobin, in said sample, is quantified,
the concentration of D-dimers in said sample is quantified,
the concentration of said cardiac marker(s) of the sample is compared to a predetermined threshold concentration,
the concentration of D-dimers of the sample is compared to a predetermined threshold concentration of D-dimers, and
it is determined whether the concentration of said cardiac marker(s) and the concentration of D-dimers of the sample are respectively less than the predetermined threshold concentration of said cardiac marker(s) and than the predetermined threshold concentration of D-dimers.

2. The method as claimed in claim 1, according to which:
the concentration of troponin in said sample is quantified,
the concentration of D-dimers in said sample is quantified,
the concentration of troponin of the sample is compared to a predetermined threshold concentration of troponin,
the concentration of D-dimers of the sample is compared to a predetermined threshold concentration of D-dimers, and
it is determined whether the concentration of troponin and the concentration of D-dimers of the sample are respectively less than the predetermined threshold concentration of troponin and than the predetermined threshold concentration of D-dimers.

3. The method as claimed in claim 2, according to which:
the concentration of troponin in said sample is quantified, then
the concentration of D-dimers in said sample is quantified,
the concentration of troponin of the sample is compared to a predetermined threshold concentration of troponin,
the concentration of D-dimers of the sample is compared to a predetermined threshold concentration of D-dimers, and
it is determined whether the concentration of troponin and the concentration of D-dimers of the sample are respectively less than the predetermined threshold concentration of troponin and than the predetermined threshold concentration of D-dimers.

4. The method as claimed in claim 2, according to which:
the concentration of D-dimers in said sample is quantified, then
the concentration of troponin in said sample is quantified,
the concentration of troponin of the sample is compared to a predetermined threshold concentration of troponin,
the concentration of D-dimers of the sample is compared to a predetermined threshold concentration of D-dimers, and
it is determined whether the concentration of troponin and the concentration of D-dimers of the sample are respectively less than the predetermined threshold concentration of troponin and than the predetermined threshold concentration of D-dimers.

5. The method as claimed in claim 2, according to which:
the concentration of troponin and the concentration of D-dimers in said sample are simultaneously quantified,
the concentration of troponin of the sample is compared to a predetermined threshold concentration of troponin,
the concentration of D-dimers of the sample is compared to a predetermined threshold concentration of D-dimers, and
it is determined whether the concentration of troponin and the concentration of D-dimers of the sample are respectively less than the predetermined threshold concentration of troponin and than the predetermined threshold concentration of D-dimers.

6. The method as claimed in claim 1, in which the liquid sample is selected from serum, plasma and blood.

7. The method as claimed in claim 1, in which the quantification of the concentration of said cardiac marker(s) and the quantification of the concentration of D-dimers in the sample are carried out, independently of one another, by means of an immunoassay.

8. The method as claimed in claim 2, in which the quantification of the concentration of troponin and the quantification of the concentration of D-dimers in the sample are carried out, independently of one another, by means of an immunoassay.

9. The method as claimed in claim 7, in which the quantification of the concentration of said cardiac marker(s) and the quantification of the concentration of D-dimers in the sample are carried out, independently of one another, on the basis of an assay selected from ELISA, ELFA, turbidimetry, nephelometry, turbidimetry with particles, nephelometry with particles, and latex agglutination.

10. The method as claimed in claim 8, in which the quantification of the concentration of troponin and the quantification of the concentration of D-dimers in the sample are carried out, independently of one another, on the basis of an assay selected from ELISA, ELFA, turbidimetry, nephelometry, turbidimetry with particles, nephelometry with particles, and latex agglutination.

11. A method for quantifying the concentration of at least one cardiac marker selected from troponin, CK-MB and myoglobin, in a liquid sample, comprises utilizing a reagent, and for quantifying the concentration of D-dimers in a liquid sample, comprises utilizing a reagent, for the in vitro diagnosis of the exclusion of an acute coronary syndrome.

12. The method, as claimed in claim 11, for quantifying the concentration of troponin in a liquid sample, comprises utilizing a reagent, and for quantifying the concentration of D-dimers in a liquid sample, comprises utilizing a reagent, for the in vitro diagnosis of the exclusion of an acute coronary syndrome.

13. The method as claimed in claim 11, in which one of the reagents is at least one antibody for at least one cardiac marker and the other of the reagents is at least one anti-D-dimer antibody.

14. The method as claimed in claim 12, in which one of the reagents is at least one anti-troponin antibody and the other of the reagents is at least one anti-D-dimer antibody.

15. The method as claimed in claim 11, in which the liquid sample is selected from serum, plasma and blood.

16. The method as claimed in claim 2, in which the liquid sample is selected from serum, plasma and blood.

17. The method as claimed in claim 12, in which the liquid sample is selected from serum, plasma and blood.

18. The method as claimed in claim 13, in which the liquid sample is selected from serum, plasma and blood.

19. The method as claimed in claim 14, in which the liquid sample is selected from serum, plasma and blood.
